(19) United States
(12) Patent Application Publication
(10) Pub. No.: US 2025/0334511 A1
(43) Pub. Date: Oct. 30, 2025

(54) METHOD OF ACQUIRING A NUTRIENT LEVEL MEASUREMENT OF A SAMPLE IN THE FIELD AND MOBILE WORKSTATION THEREFOR

(71) Applicant: PICKETA SYSTEMS INC., Fredericton (CA)

(72) Inventors: Maxime DUMONT, Dieppe (CA); Zachary ANDERSEN, Edmundston (CA)

(21) Appl. No.: 18/649,033

(22) Filed: Apr. 29, 2024

(51) Int. Cl. G01N 21/25; B01L 1/00; G01N 33/02

(52) U.S. Cl. CPC G01N 21/255; B01L 1/52; G01N 33/025; B01L 2200/18; B01L 2300/027; G01N 2201/0221; G01N 2201/127

(57) ABSTRACT

There is disclosed a method of performing a nutrient level measurement for a crop in the field using a mobile workstation, the mobile workstation having a casing containing a spectrometer, a probe and a planar worksurface. The method includes carrying the casing to a crop location in the field, opening the casing, thereby exposing the planar worksurface, performing a plurality of scans of the crop, including, for each one of the scans severing a sample from the crop, positioning the sample of the crop onto the exposed planar worksurface, positioning the probe onto the sample, on the planar worksurface, acquiring a spectrum of the sample with the spectrometer, via the probe, and removing the sample from the planar worksurface and subsequently to said performing the plurality of scans, closing the casing.

### FIELD

The improvements generally relate to crop analysis, and more specifically to a method of performing a nutrient level measurement of a crop in a field using a mobile workstation.

### BACKGROUND

Agriculture makes a significant use of fertilizers for reasons such as optimizing yield. Fertilizers consist of nutrients that the crops need to develop to their full potential. There are various types of nutrients and different types of crops that may need different amounts of different types of nutrients. While the nutrients are typically already present in the ground to a certain extent, the amount present naturally in the ground is typically insufficient to allow the crops to reach their full potential or optimized yield. However, while fertilizers have significant benefits in agriculture, they are also a significant source of cost. Providing an excessive amount of fertilizer to soil where crops grow can thus represent a loss of profit for the farmers, in addition to potentially having negative effects on the environment.

There is thus a motivation for farmers to provide not only enough fertilizers for their crops, but also just enough fertilizers for their crops. While this objective may appear simple at first glance, there are various challenges to achieving it in practice, which can lead to excessive or insufficient use of fertilizers. Indeed, to provide “just enough” fertilizers for their crops, farmers need to know how much fertilizer their crops need. Farmers, based on their experience and just by looking at them, can sometimes tell that a given crop would benefit from a certain amount of a certain type of fertilizer. However, in practice, this method is often inaccurate. An alternative is to take samples of the crop in the field, and to bring these samples to a laboratory which may take measurements of the levels/concentration of nutrients in the samples. While this may lead to a greater accuracy than a farmer's experience-based assessment, there may be a deterioration of the sample between the harvesting of the sample and the moment when the measurement is made, which may bias the results and introduce a source of inaccuracy. Moreover, this process is relatively tedious and time-consuming. For instance, nutrient concentrations can vary depending on the location on the field, and to be relevant, several samples may need to be taken from different locations on the field. There can be confusion, once the results of the laboratory analysis are received, as to which results correspond to which location on the field. Indeed, different samples may become mixed up during collection or during transport, or even during testing, and the process of correctly grouping and identifying samples can be tedious and cumbersome, let alone the inconveniences of the transport and of the delay between the collection of the samples and the receipt of the analysis results. The delay between sending samples to the laboratory and receiving results thus prevents timely fertilizer applications, as in this case the needs of the crop at the moment where the results are received do not necessarily match the needs of the crop at the moment where the samples were taken. Other instruments, such as ones based on chemical analysis, require multiple individual sensors each measuring a respective type of nutrient.

Accordingly, while known techniques of obtaining indications of the nutrient levels of the crop were satisfactory to a certain degree, there remains significant room for improvement, and such improvements would likely be correlated to a better use of fertilizers, such as a reduction in the use of fertilizers and associated cost savings and environmental benefits, or an increase in the use of fertilizers which would have a direct effect on yield and thus be a source of profit.

### SUMMARY

One potential technique for performing nutrient level measurement is via spectral analysis. Spectral analysis can involve the acquisition of a spectrum of the crop which can be performed via a spectrometer. Spectrometers are specialized pieces of equipment and typically need to be used by trained technicians, as in particular, image quality may depend strongly on how closely a calibration protocol was followed and/or how the spectrometers are used. Spectrometers are thus not configured to be easily carried around, and especially in a field, where particles and other debris may interfere with the readings and damage the optical and/or electronic components.

It was found that at least in some embodiments, and when appropriately guided by certain hardware elements and a dynamic user interface, it was possible for average farmers to acquire spectra of good quality and reliability, thereby avoiding the need for manually labelling, handling, and transporting the samples between the field and the laboratory. This opened new possibilities, while nonetheless leaving open the question of how to make the hardware convenient to use in the intended context of use.

It was found that providing the hardware in a way which allowed carrying it to a plurality of locations on the field, and to take the nutrient level measurements at the locations of the crops, was the most promising avenue to make the technology convenient and achieve user buy-in. It was found that this could be achieved by providing the hardware in the form of a mobile workstation. More specifically, the mobile workstation may be provided as a casing carriable by the farmer in one hand, implying that the casing is sized and shaped to be relatively easy to carry by hand, like a suitcase. When the farmer arrives at a location in the field where the crop samples are located, the farmer can then gain access to the sensitive instrumentation contained inside, and to a conveniently positioned planar work surface, by opening the casing. In other implementations, the farmer can also drive around the field in a vehicle, and open the workstation on the bed of a truck, for instance, or on the passenger seat. Once the spectral measurements are acquired, the farmer can close the casing, thereby protecting the sensitive equipment and the planar worksurface from the hazards of the field such as precipitation and dirt, and move to another location for analyzing other crop samples. Such mobile workstation is therefore a suitable solution to performing spectral measurements in the field, at least in some embodiments.

In accordance with a first aspect of the present disclosure, the is presented a method of performing a nutrient level measurement for a crop in the field using a mobile workstation, the mobile workstation having a casing containing a spectrometer, a probe and a planar worksurface, the method comprising carrying the casing to a crop location in the field, opening the casing, thereby exposing the planar worksurface, performing a plurality of scans of the crop, including, for each one of the scans severing a sample from the crop, positioning the sample of the crop onto the exposed planar worksurface, positioning the probe onto the sample, on the planar worksurface, acquiring a spectrum of the sample with the spectrometer, via the probe, and removing the sample from the planar worksurface and subsequently to said performing the plurality of scans, closing the casing.

In some embodiments, the crop location includes a plurality of locations in and each of said carrying the casing, opening the casing, performing the plurality of scans of the crop and closing the casing is performed for each location of the plurality of locations.

In some embodiments, said performing the plurality of scans of the crop includes following commands or indications on a display screen.

In some embodiments, said carrying the casing includes hand-carrying the casing, further comprising laying the casing on the ground prior to said opening the casing.

In some embodiments, said carrying the casing includes carrying the casing in a vehicle, further comprising laying the casing onto a horizontal surface of the vehicle prior to said opening the casing.

In some embodiments, said performing the plurality of scans of the crop includes calibrating a spectrometer and said acquiring the spectrum of the sample includes placing a probe connected with the spectrometer over the sample.

In some embodiments, the casing has a first half and a second half hinged to one another, and said opening the casing includes pivoting the first half relative to the second half.

In some embodiments, the mobile workstation further comprises a fiber optic cable connecting the probe to the spectrometer.

In accordance with a second aspect of the present disclosure, there is presented a mobile workstation for acquiring a spectrum of a sample, the mobile workstation comprising a casing having a top portion and a bottom portion, the casing being switchable between a closed configuration in which the top portion is attached to the bottom portion and an interior of the top portion faces an interior of the bottom portion and an opened configuration in which the interior of the top portion and the interior of the bottom portion are exposed, a planar worksurface integrated to the bottom portion, the planar worksurface defining a sample receiving area and being parallel to the working surface and a spectrometer integrated to the casing.

In some embodiments, the mobile workstation further comprises a display screen configured for displaying commands or indications to a user.

In some embodiments, the mobile workstation further comprises a computing device having a processing unit and a non-transitory computer-readable memory having stored thereon program instructions executable by the processing unit for: providing signals representative of the commands or indications to the display screen and receiving the spectrum obtained by the spectrometer.

In some embodiments, the mobile workstation further comprises a probe and an fiber optic cable connecting the probe with the spectrometer.

In some embodiments, the bottom portion of the casing is configured to be abutted to the ground.

In some embodiments, the interior of the top portion faces away from the interior of the bottom portion in opened position.

In some embodiments, an extremity of the top portion is attached to an extremity of the bottom portion via a hinge portion in the opened position.

In some embodiments, the spectrometer is integrated in one of the top portion and the bottom portion.

In some embodiments, the spectrometer is integrated underneath the planar worksurface.

In some embodiments, the bottom portion defines an annular cavity for coiling a portion of the fiber optic cable.

In some embodiments, the spectrometer comprises a plurality of spectrometers each covering a respective spectral band.

In some embodiments, the top portion is sealingly attached to the bottom portion in the closed position.

Many further features and combinations thereof concerning the present improvements will appear to those skilled in the art following a reading of the instant disclosure.

### DESCRIPTION OF THE FIGURES

In the figures,

FIG. 1 is a diagram showing a user acquiring a nutrient level of a sample in a field using a mobile workstation, in accordance with one embodiment;

FIG. 2 is a diagram showing a user acquiring a nutrient level of a sample in a vehicle using a mobile workstation, in accordance with one embodiment;

FIG. 3 is a schematic diagram of the mobile workstation of FIGS. 1 and 2, in accordance with one embodiment;

FIG. 4 is a perspective view of an insert panel insertable in a bottom portion of the mobile workstation of FIG. 3, in accordance with one embodiment;

FIG. 5 is a top view of an insert panel insertable in a bottom portion of the mobile workstation of FIG. 3, in accordance with one embodiment;

FIG. 6 is a flowchart of a method of acquiring a spectrum using the mobile workstation of FIG. 3, in accordance with one embodiment; and

FIG. 7 is a block diagram illustrating an example computing device, in accordance with one embodiment.

### DETAILED DESCRIPTION

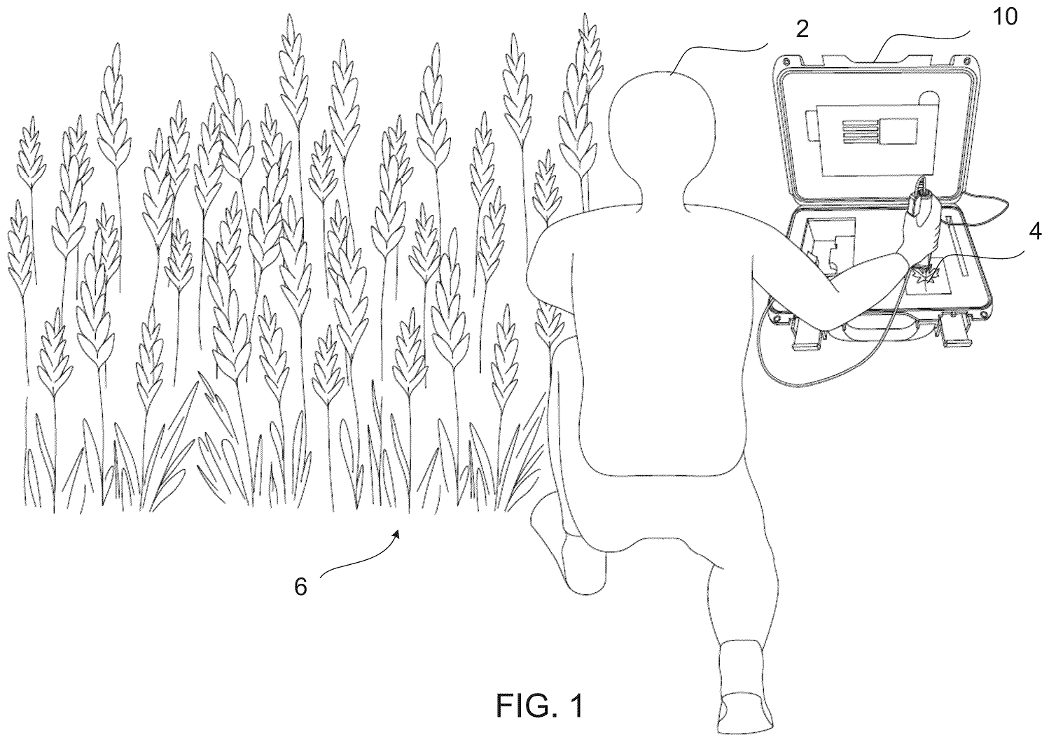

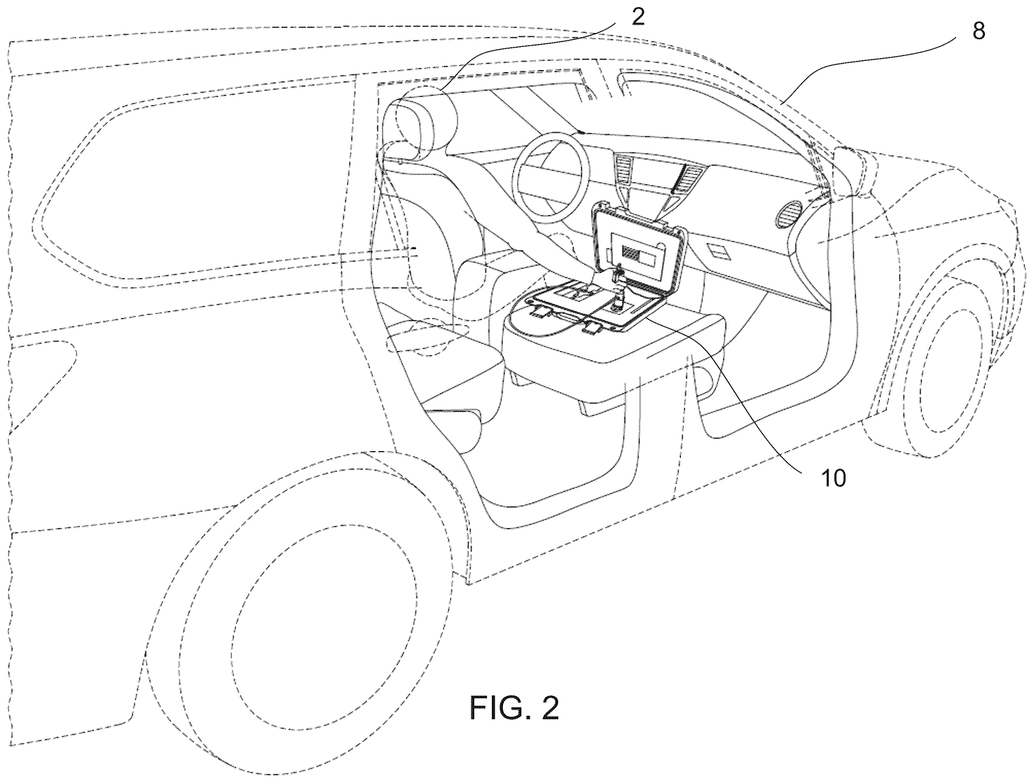

Described below are exemplary methods of acquiring a nutrient level measurement of a sample in the field, along with a mobile workstation for acquiring the nutrient level. Referring to FIGS. 1 and 2, there is shown a user 2 acquiring a nutrient level of a crop sample 4 in a field 6 or in a vehicle 8 using a mobile workstation 10. The mobile workstation 10 includes a casing containing a spectrometer, a probe, a fiber optic cable connecting the probe to the spectrometer, and a planar worksurface. The mobile workstation 10 will be described in more detail further below.

In operation, the user 2 carries the mobile workstation 10 to a crop location around in the field 6. As such, the mobile workstation 10 can be hand-carried around in the hand of the user 2, or on a horizontal surface of the vehicle, such as the bed, the trunk or a passenger seat. It will be appreciated that a field 6 is usually a vast place where numerous crops are grown, and where the crops are grouped by criteria, such as the type of crop, the year it was planted, and the like. As such, in operation, the user 2 carries the mobile workstation 10 around the field 6 at various locations for analyzing various types of crop samples 4. In some embodiments, once arrived at a crop location, as depicted in FIG. 1, the user 2 may lay the mobile workstation 10 on the ground or on a planar surface, such as a table. In other embodiments, as depicted in FIG. 2, the user may drive around in a vehicle 8, such as a car, a pick-up truck, a tractor, and the like, and lay the mobile workstation 10 on the passenger seat or in the bed or trunk of the vehicle 8.

Once the mobile workstation 10 is in position near the field 6 or in the vehicle 8, the user 2 may open the casing of the mobile workstation 10, thereby exposing a planar worksurface. It will be appreciated that the planar worksurface may be configured to receive the crop sample so that spectral measurements can be performed thereon. The mobile workstation 10 is openable by separating two portions from one another. In some cases where the casing has a front-opening mechanism, the mobile workstation 10 may be opened by unlocking the casing, in the case where the casing is locked, and pivoting a first half relative to a second half, the first half and the second half being attached via a hinge portion. In some embodiments, the first half may be pivoted to an angle of about 90 degrees or higher relative to the second half. In some embodiments, a means to retain the first half relative to the second half for a given angle may be included in the hinge portion, said means including a torsion spring, for instance. It will be appreciated that the mobile workstation 10 is not limited to a front-opening mechanism, as other types of mechanisms may apply, such as a side-opening mechanism.

After opening the mobile workstation 10, the user 2 may perform a plurality of scans of the crop that is at the location where the mobile workstation 10 is positioned. For each of the scans, the user may sever the sample 4 from the crop. The sample 4 may be, but not limited to, a leaf, a stem, a root, a spike, an awn, and the like. It will be appreciated that, in some cases, the sample 4 of the crop may be severed prior to placing and opening the mobile workstation 10. Once severed, the sample 4 of the crop may be positioned onto the exposed planar worksurface of the mobile workstation 10. In some embodiments, the sample 4 may be placed on a sample receiving area that is parallel to the working surface, the sample receiving area having spectral properties suited for spectral measurements. For instance, the sample receiving area may have an absorption value in the spectral bands measured by the spectrometer, which can help distinguish the spectral signal of the sample from the spectral signal of the sample receiving area. Once the sample 4 is placed on the planar worksurface, the user 2 may acquire a spectrum of the sample 4 using a spectrometer, via a probe connected thereto. This spectrum acquisition may be reperformed for a plurality of iterations, in accordance with the embodiment. After acquiring the spectrum, the sample 4 may be removed from the planar worksurface, and be discarded in the field 6 or be stored for further measurements.

Subsequently to the performing of the plurality of scans of the sample 4, the user 2 may close the casing of the mobile workstation 10, and may either stop measuring crop samples or repeat the measurements of crop samples at other locations in the field 6.

In some embodiments, the user 2 may follow commands or indications on a display screen of the mobile workstation when performing the plurality of scans of the crop. The commands or indications may include performing a calibration sequence prior to acquiring the spectrum of the sample 4.

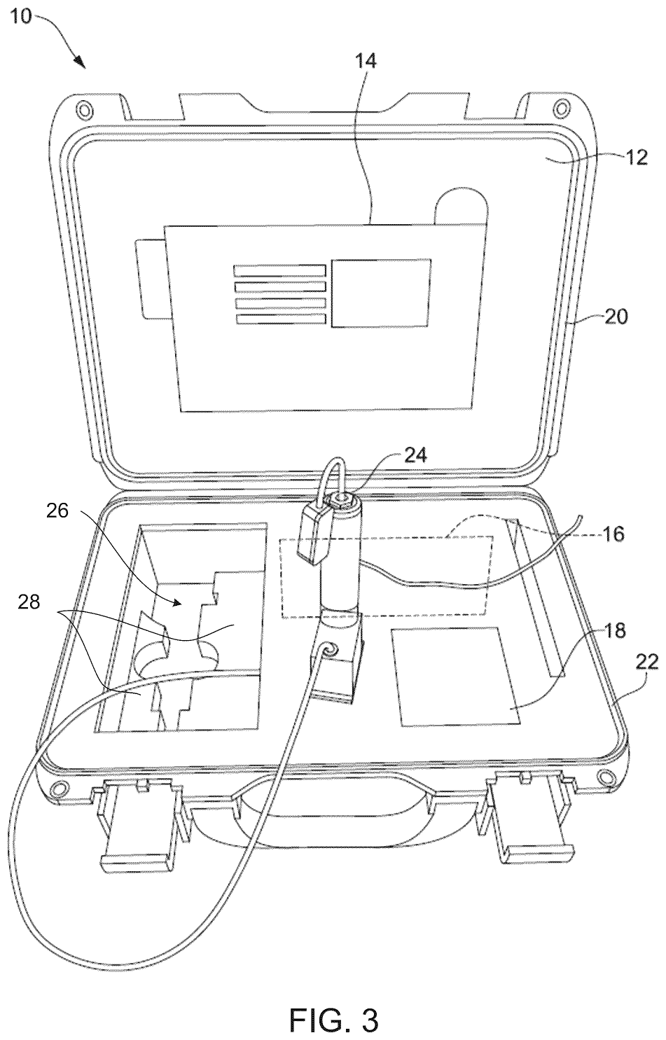

Referring now to FIG. 3, there is shown a front view of a mobile workstation 10. The mobile workstation 10 includes a casing 12 containing a computing device 14, a spectrometer 16 and a sample receiving area 18. The computing device 14 may include a user interface. The user interface may include one or more elements depending on the embodiment, and may include, for instance, a display screen, a touch screen, an audible signal emitter, a visual signal emitter, a keyboard, a mouse, etc. In the example presented, the user interface includes a display device that may be configured to display a graphical user interface (GUI). The computing device 14 further includes a processing unit and a non-transitory computer-readable memory having stored thereon program instructions executable by the processing unit for acquiring spectra of crop samples. The casing 12 may be composed of a top portion 20 that houses the computing device 14 and a bottom portion 22 that houses the spectrometer 16 and defines the sample receiving area 18 on a planar worksurface. It will be appreciated that the computing device 14 is not limited to be integrated to the top portion 20, and can be located elsewhere at the disposition of the user. Further, it will be appreciated that the spectrometer 16 is not limited to be integrated to the bottom portion 22, and can be located elsewhere at the disposition of the user.

The casing 12 is switchable between a closed configuration in which the top portion 20 is attached to the bottom portion 22 and an interior of the top portion 20 faces an interior of the bottom portion 22, and an opened configuration in which the interior of the top portion 20 and the interior of the bottom portion 22 are exposed. In some cases, an extremity of the top portion 20 and an extremity of the bottom portion 22 are attached via a hinge portion so that the casing 12 can be opened for use. In this case, in the opened configuration, the top portion 20 is upright and faces the user, such that the display of the computing device 14 is placed in front of the user. The bottom portion 22 can be placed on the ground and planar therewith such that the sample receiving area 18 is parallel with the ground. It will be appreciated that while the mobile workstation 10 is generally suited to be used in the field where crops are grown, other areas should be contemplated in which the mobile workstation 10 is used for similar purposes, such as in a greenhouse, a laboratory, an indoor field and any other suited area. In some embodiments, the interior of the top portion 20 faces away from the interior of the bottom portion 22 in opened position

The display device of the computing device 14 can be embedded within the top portion 20 of the casing, and can include a touchscreen for the user to be able to interact with the computing device 14. The computing device 14 may alternately be equipped with a mouse or other pointer-type input devices for interacting with the user. In operation, the computing device 14, may be configured to prompt the user for scanning a crop sample by placing the sample on the sample receiving area 18 and scanning the sample with a probe 24 coupled with the spectrometer 16. The output from the spectrometer in that case is generally referred to herein as a spectrum of the sample. In some embodiments, a probe 24 suited for the mobile workstation 10 is an instrument configured to position the end of a fiber optic cable at a preferred angle and distance with respect to the sample when performing scans. The probe 24 can also be equipped with a lamp for illuminating the sample with light having spectral bands that correspond to the spectral bands of the spectrometer. The probe 24 may define an opening for the light generated by the lamp to be provided to the sample, and for the light obtained from the sample to be provided to the spectrometer 16. In some embodiments the light can be guided with fiber optic cables between the probe 24, the lamp and the spectrometer 16.

In some embodiments, the bottom portion 22 defines a cavity 26 in which the probe 24 and the fiber optic cable attached to the probe 24 can be stored when the casing 12 is in the closed position. Inside the cavity, blocks 28 matching the shape of the probe 24 can be fixed to the bottom portion 22 to receive the probe 24 and to prevent the probe 24 from moving when handling the mobile workstation 10. The blocks 28 may be composed of foam or a similar material.

It will be appreciated that a fiber optic cable is an optoelectronic component that contains one or more optic fibers therein, which can be surrounded by a protective sheath. In some embodiments, the fiber optic cable contains a single optic fiber. In operation and when attached to the probe 24, the light reflected from the sample enters each of the optical fibers of the fiber optic cable and travels towards the spectrometer 16.

A spectrometer 16 suited for the application is able to cover the spectral band defined between about 350 nm and 2,500 nm, and may thus include measurements of wavelengths in the near-infrared portion of the electromagnetic spectrum in addition to wavelengths in the visible portion of the electromagnetic spectrum. It will be appreciated that some spectrometers may not cover such a large band. As such, two, or more spectrometers 16 having non-overlapping or partially non-overlapping bandwidths may be used complementarily to cover the desired spectral bands. In some cases, the spectral band of interest is defined between about 350 nm and 1,700 nm. In some other cases, the spectral band of interest is defined in the mid-infrared (MIR). The term spectrum generally refers to a spectral measurement of the crop sample. It will be appreciated that the spectroscopy technique used to obtain the spectra may vary, and may include, but is not limited to, near-infrared (NIR) spectroscopy, MIR spectroscopy, Raman spectroscopy, UV-visible spectroscopy, and/or the combination thereof. According to the technique, the presence of various types of nutrients can be detected, such as, but not limited to, nitrogen (N), phosphorus (P), potassium (K), and the like.

In some embodiments, the spectrum may be a spectral image, which is a bidimensional image of a crop sample in which wavelengths outside the visible spectrum may also be captured. The spectrum may include spatial distribution information, e.g. more than one pixel, and in practice, the spectrum may include a large number of pixels. In an alternate embodiment, the spectrum may not include spatial distribution information and only a blended amplitude distribution covering various wavelengths within the bandwidth. In some embodiments, the probe 24 may be configured to scan a 2D surface of the crop sample, using rasterization or other suited techniques. It will be appreciated that the term “scan” referred to herein is representative of causing the spectrometer to measure a spectrum of the sample.

The sample receiving area 18 is generally defined by a surface with low reflectivity across the spectral band of interest. As such, the sample receiving area 18 usually generates low signal when scanning a sample with the probe 24. The sample receiving area 18 is generally sized and shaped for receiving a leaf or a crop sample of similar dimensions. In operation, the user places the sample onto the sample receiving area 18 and scans the sample using the probe 24. While the sample receiving area 18 is defined on the bottom portion 22 of the casing, in some embodiments, the sample receiving area 18 may also be placed outside the casing, as a separate piece of equipment.

It will be appreciated that the mobile workstation 10 is equipped with suited power supply, such as a rechargeable battery, to be carried around a field and be powered when operating. In some embodiments, the casing 12 includes a handle and is generally sized and shaped to be carried around a field by a single user. While the display device of the computing device 14 is preferred on the top portion 20 of the casing 12, the other hardware components can be placed elsewhere in the casing 12, or, in some cases, outside of the casing 12.

In some embodiments, the planar worksurface of the mobile workstation 10 defines a calibration sample receiving area (not shown) being parallel to the planar worksurface. The calibration samples are generally samples with predetermined spectral properties that are used to obtain the integration time and the baseline for the spectrometers. In some cases, the calibration sample receiving area includes a magnetized element releasably attachable with a corresponding magnetized element attached to the calibration sample. In some embodiments, the calibration sample receiving area can be the sample receiving area 18.

In some embodiments, the top portion 20 and the bottom portion 22 are sealingly attached with one another in the closed position, thereby preventing contaminants from the environment to enter the casing 12. In some cases, a gasket made of a sealing material, such as rubber or silicon, is attached to the edges of the interior of the top portion 20 and/or the interior of the bottom portion 22.

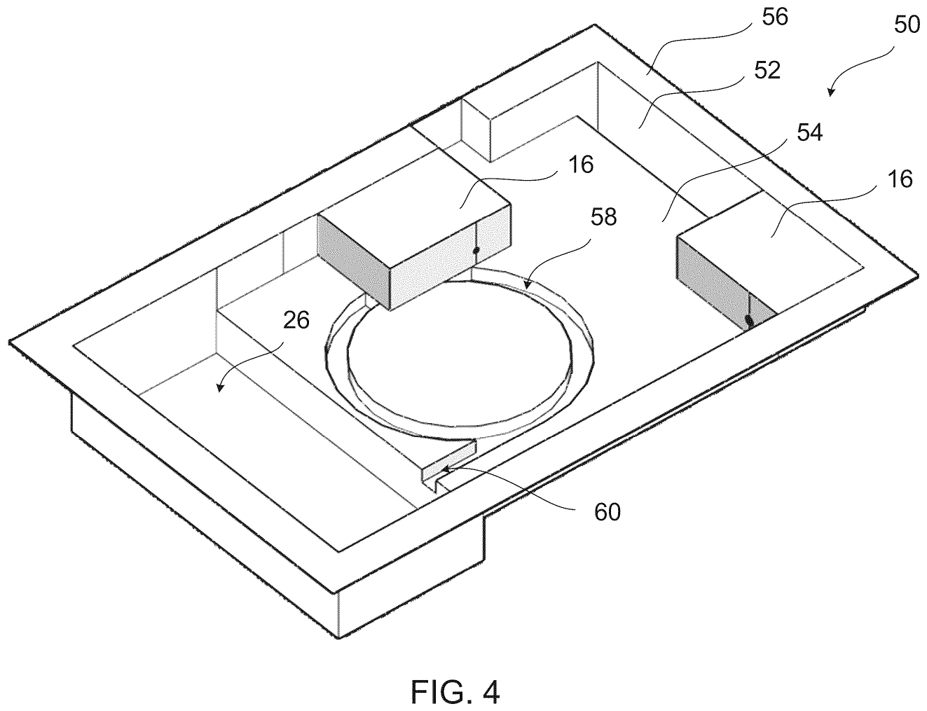

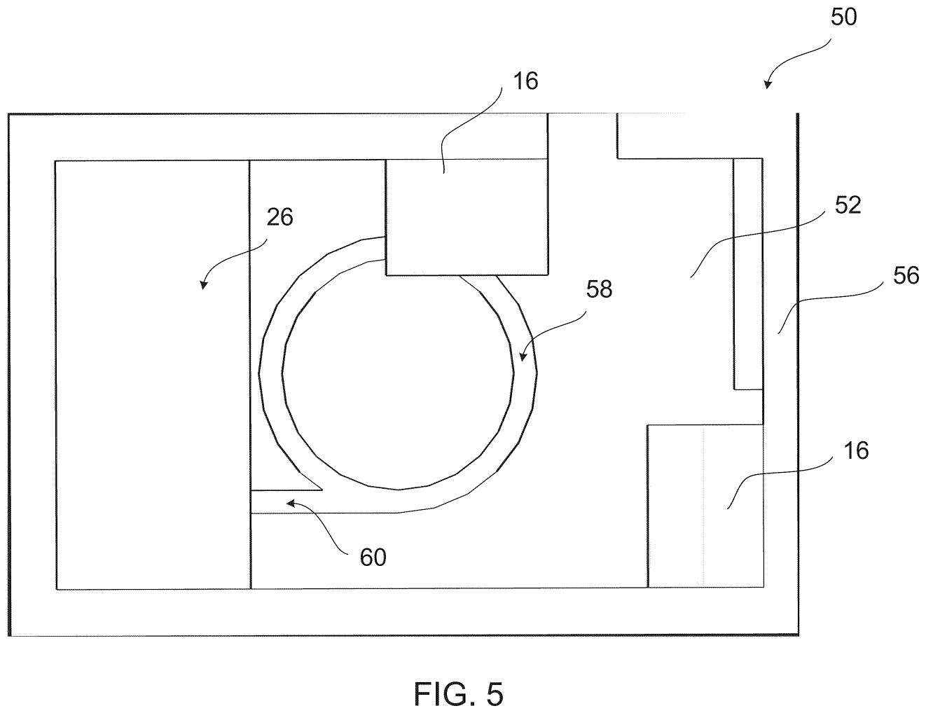

Referring now to FIGS. 4 and 5, there are shown a perspective view and a top view, respectively, of an insert panel 50 insertable in the bottom portion 22 of the mobile workstation 10, beneath the planar worksurface. The insert panel 50 has lateral walls 52, a bottom wall 54 and a ledge 56 that extends outwardly from an upper portion of the lateral walls 52. The bottom wall 54 also defines a recessed portion in which the cavity 26 for storing the probe 24 is located. Once placed inside the bottom portion 22, a bottom surface of the ledge 56 abuts a matching surface (not shown) of the bottom portion 22, so that the insert panel 50 stays in place when inserted in the bottom portion 22. In some embodiments, the insert panel 50 is secured to the bottom portion 22 via suited fasteners.

As shown in FIGS. 4 and 5, two spectrometers 16 are attached to the lateral walls 52. A fiber optic cable may be connected to each of the spectrometers 16, and the two fiber optic cables may be coupled together using an cable connector. In that case, an output of the optic cable connector can be connected to the probe 24, such that the light captured by the probe can be provided to both spectrometers 16 via the cable connector. The connector can be configured to redirect any optical fiber contained in the input fiber optic cable towards the output fiber optic cables. For instance, and in accordance with one embodiment, the fiber optic cable connected to the probe 24 may include seven optical fibers. The cable connector may redirect three of those optical fibers towards one spectrometer, and the remaining four towards the other spectrometer.

In some embodiments, only a single spectrometer 16 can be used and is therefore coupled to the probe 24. In some cases, the bottom wall 54 defines an annular cavity 58 for coiling the fiber optic cable connected to the probe 24. The annular cavity 58 is defined by an inner diameter, an outer diameter and a depth. It will be appreciated that the cross-section of the annular cavity 58 is generally larger than the cross-section of the fiber optic cable, so that the fiber optic cable can be coiled inside the annular cavity 58. In some cases, the annular cavity 58 is sized and shaped to receive a plurality of coils of the fiber optic cable. Furthermore, it will be appreciated that the inner diameter of the annular cavity 58 is generally chosen to avoid damaging the fiber optic cable and avoid generating bending losses in the signal. In some embodiments, the bottom wall 54 defines a canal 60 connecting the annular cavity 58 to the cavity 26 for securing the portion of the fiber optic cable exiting the annual cavity 58 towards the probe 24. It will be appreciated that the larger the number of coils are placed in the annular cavity 58, the longer the length of the fiber optic cable connecting the probe 24 to the spectrometers 16 will be. In some embodiments, the stray light not collected in the optical fibers of the fiber optic cable may induce noise in the signal produced by the spectrometer 16. Coiling the fiber optic cable may reduce the presence of such stray light in the light provided to the spectrometer 16 by trapping the stray light in the coil.

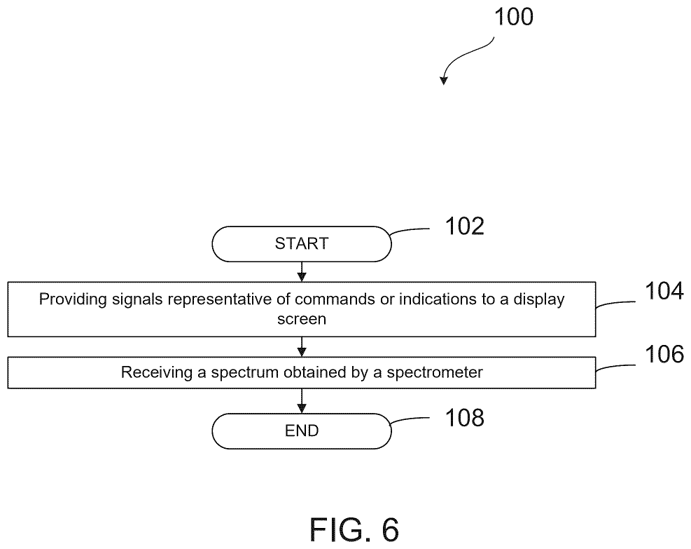

Now referring to FIG. 6, there is shown a flowchart of a method 600 for acquiring a spectrum of a crop sample using the mobile workstation 10. The method 100 starts at step 102. At step 104, signals representative of commands or indications are provided to a display screen. It will be appreciated that the commands or indications displayed on the screen are provided to guide the user to perform spectral measurements on the crop samples. The commands or indications may prompt the user to perform predetermined actions, such as, but not limited to, waiting for a lamp illuminating the spectrometers to warm up, calibrating the spectrometers, placing the probe over the sample, interacting with the UI once the probe is placed for acquiring the spectrum, and the like. In response to said providing the signals, at step 106, a spectrum obtained by the spectrometer is received. The method 100 continues at step 108.

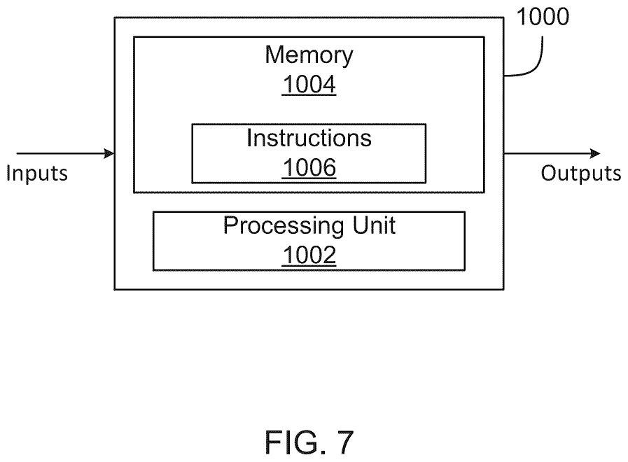

Referring now to FIG. 7, the method 100 of FIG. 6, may be implemented using a computing device 1000. For simplicity only one computing device 1000 is shown but the method 100 may involve more computing devices 1000 which may be the same or different types of devices. The computing device 1000 comprises a processing unit 1002 and a memory 1004 which has stored therein computer-executable instructions 1006. The processing unit 1002 may comprise any suitable devices configured to implement the method 100 such that instructions 1006, when executed by the computing device 1000 or other programmable apparatus, may cause the functions/acts/steps of the method 100 described herein to be executed. The processing unit 1002 may comprise, for example, any type of general-purpose microprocessor or microcontroller, a digital signal processing (DSP) processor, a central processing unit (CPU), an integrated circuit, a field programmable gate array (FPGA), a reconfigurable processor, other suitably programmed or programmable logic circuits, or any combination thereof.

The memory 1004 may comprise any suitable known or other machine-readable storage medium. The memory 1004 may comprise non-transitory computer readable storage medium, for example, but not limited to, an electronic, magnetic, optical, electromagnetic, infrared, or semiconductor system, apparatus, or device, or any suitable combination of the foregoing. The memory 1014 may include a suitable combination of any type of computer memory that is located either internally or externally to device, for example random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Memory 1004 may comprise any storage means (e.g., devices) suitable for retrievably storing machine-readable instructions 1006 executable by processing unit 1002.

The above description is meant to be exemplary only, and one skilled in the art will recognize that changes may be made to the embodiments described without departing from the scope of the invention disclosed. Still other modifications which fall within the scope of the present invention will be apparent to those skilled in the art, in light of a review of this disclosure.

Various aspects of the systems and methods described herein may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments. Although particular embodiments have been shown and described, it will be apparent to those skilled in the art that changes and modifications may be made without departing from this invention in its broader aspects. The scope of the following claims should not be limited by the embodiments set forth in the examples, but should be given the broadest reasonable interpretation consistent with the description as a whole.

What is claimed is:

1. A method of performing a nutrient level measurement for a crop in the field using a mobile workstation, the mobile workstation having a casing containing a spectrometer, a probe, and a planar worksurface, the method comprising:
carrying the casing to a crop location in the field;
opening the casing, thereby exposing the planar worksurface;
performing a plurality of scans of the crop, including, for each one of the scans:
severing a sample from the crop,
positioning the sample of the crop onto the exposed planar worksurface,
positioning the probe onto the sample, on the planar worksurface,
acquiring a spectrum of the sample with the spectrometer, via the probe, and
removing the sample from the planar worksurface; and
subsequently to said performing the plurality of scans, closing the casing.

2. The method of claim 1, wherein the crop location includes a plurality of locations in and each of said carrying the casing, opening the casing, performing the plurality of scans of the crop and closing the casing is performed for each location of the plurality of locations.

3. The method of claim 1, wherein said performing the plurality of scans of the crop includes following commands or indications on a display screen.

4. The method of claim 1, wherein said carrying the casing includes hand-carrying the casing, further comprising laying the casing on the ground prior to said opening the casing.

5. The method of claim 1, wherein said carrying the casing includes carrying the casing in a vehicle, further comprising laying the casing onto a horizontal surface of the vehicle prior to said opening the casing.

6. The method of claim 1, wherein said performing the plurality of scans of the crop includes calibrating a spectrometer and said acquiring the spectrum of the sample includes placing a probe connected with the spectrometer over the sample.

7. The method of claim 1, wherein the casing has a first half and a second half hinged to one another, and said opening the casing includes pivoting the first half relative to the second half.

8. The method of claim 1, wherein the mobile workstation further comprises a fiber optic cable connecting the probe to the spectrometer.

9. A mobile workstation for acquiring a spectrum of a sample, the mobile workstation comprising:
a casing having a top portion and a bottom portion, the casing being switchable between:
a closed configuration in which the top portion is attached to the bottom portion and an interior of the top portion faces an interior of the bottom portion; and
an opened configuration in which the interior of the top portion and the interior of the bottom portion are exposed;
a planar worksurface integrated to the bottom portion, the planar worksurface defining a sample receiving area and being parallel to the working surface; and
a spectrometer integrated to the casing.

10. The mobile workstation of claim 9, further comprising a display screen configured for displaying commands or indications to a user.

11. The mobile workstation of claim 10, further comprising a computing device having a processing unit and a non-transitory computer-readable memory having stored thereon program instructions executable by the processing unit for:
providing signals representative of the commands or indications to the display screen; and
receiving the spectrum obtained by the spectrometer.

12. The mobile workstation of claim 9, further comprising a probe and an fiber optic cable connecting the probe with the spectrometer.

13. The mobile workstation of claim 9, wherein the bottom portion of the casing is configured to be abutted to the ground.

14. The mobile workstation of claim 9, wherein the interior of the top portion faces away from the interior of the bottom portion in opened position.

15. The mobile workstation of claim 14, wherein an extremity of the top portion is attached to an extremity of the bottom portion via a hinge portion in the opened position.

16. The mobile workstation of claim 9, wherein the spectrometer is integrated in one of the top portion and the bottom portion.

17. The mobile workstation of claim 16, wherein the spectrometer is integrated underneath the planar worksurface.

18. The mobile workstation of claim 12, wherein the bottom portion defines an annular cavity for coiling a portion of the fiber optic cable.

19. The mobile workstation of claim 9, wherein the spectrometer comprises a plurality of spectrometers each covering a respective spectral band.

20. The mobile workstation of claim 9, wherein the top portion is sealingly attached to the bottom portion in the closed position.
